(19) United States
(12) Patent Application Publication
(10) Pub. No.: US 2026/0306508 A1
(43) Pub. Date: Oct. 8, 2026

(54) MISOPROSTOL FORMULATION FOR NON-ORAL ADMINISTRATION

(71) Applicant: Conceptra Biosciences, LLC, Wilmington, NC (US)

(72) Inventors: Thomas W. Leonard, Wilmington, NC (US); Gary Gamerman, Vienna, VA (US); Colleen Johns, Castle Hayne, NC (US)

(73) Assignee: Conceptra Biosciences, LLC, Wilmington, NC (US)

(21) Appl. No.: 19/300,336

(22) Filed: Aug. 14, 2025

Related U.S. Application Data
(60) Provisional application No. 63/686,230, filed on Aug. 23, 2024.

(51) Int. Cl. A61K 31/5575; A61K 9/00; A61K 9/20; A61K 31/4196; A61K 31/519; A61K 31/567; A61K 31/573

(52) U.S. Cl. CPC A61K 31/5575; A61K 9/006; A61K 9/2013; A61K 9/2054; A61K 9/2059; A61K 31/4196; A61K 31/519; A61K 31/567; A61K 31/573

(57) ABSTRACT

A dispersible solid pharmaceutical composition non-oral dosage form is provided that results in a better pharmacokinetic profile in conjunction with a reduced incidence of adverse side effects and may be administered under fasting conditions. The non-oral dosage form may also have reduced oral irritation as compared to oral forms. The non-oral dosage form may include misoprostol or a pharmaceutically acceptable salt thereof, one or more disintegrants, lubricants, diluents, and optionally one or more other excipients (including but not limited or antioxidant, color, or flavor). The non-oral dosage form may have a hardness of about 6 to about 10 kp, allows administration non-orally, namely sublingually, vaginally or rectally; disintegration or dispersion in less than about 60 seconds; and has a greater than about 80 percent dissolution at 15 minutes.

### RELATED APPLICATION

The present application claims priority to U.S. Provisional No. 63/686,320 filed Aug. 23, 2024 the disclosures of which are incorporated herein by reference in their entireties.

### FIELD

The present invention relates to a solid pharmaceutical formulation comprising misoprostol or a pharmaceutically acceptable salt thereof. The dosage form of the misoprostol or pharmaceutically acceptable salt may be suited for obstetric and gynecological usages such as cervical ripening, cervical, transcervical and uterine procedures, induction of labor, prevention or treatment of postpartum hemorrhage, treatment of miscarriage and alone and concomitantly with other drugs including mifepristone, methotrexate and ulipristal acetate.

### BACKGROUND

Misoprostol is a synthetic analogue of a naturally occurring prostaglandin E₁. The formula for misoprostol is (±) methyl 11,16-dihydroxy-16-methyl-9-oxorpost-13-en-1-oate. Misoprostol is typically water soluble and water labile. Various routes of administration for misoprostol have been contemplated including oral, vaginal, sublingual, and rectal routes. Misoprostol is a drug that undergoes extensive first pass metabolism, thus non-oral routes are often used to avoid initial liver metabolism. Plasma levels of misoprostol are below the limit of detection for current analytical technologies, and absorption is monitored by measuring misoprostic acid. Variable absorption is also a concern when taken orally. The prior art alleges that the bioavailability and rate of absorption of oral tablets is reduced when taken by patients in a “fed” state as compared to a “fasted” state. The prior art also alleges that administration in the fed state reduces side effects. Frye L J, Byrne M E, Winikoff B. A crossover pharmacokinetic study of misoprostol by the oral, sublingual and buccal routes. The European Journal of Contraception & Reproductive Health Care. 2016; 21(4):265-268.

Misoprostol is a synthetic analogue of a naturally occurring prostaglandin E₁. The formula for misoprostol is (±) methyl 11,16-dihydroxy-16-methyl-9-oxorpost-13-en-1-oate. Misoprostol is typically water soluble and water labile. Various routes of administration for misoprostol have been contemplated including oral, vaginal, sublingual, buccal, and rectal routes. Misoprostol is a drug that undergoes extensive first pass metabolism, thus non-oral routes are often used to avoid initial liver metabolism. Variable absorption is also a concern when taken orally. The prior art alleges that the bioavailability and rate of absorption of oral tablets is reduced when taken by patients in a “fed” state as compared to a “fasted” state. The prior art, however, also alleges that administration in the fed state reduces side effects. Frye U, Byrne M E, Winikoff B. A crossover pharmacokinetic study of misoprostol by the oral, sublingual and buccal routes. The European Journal of Contraception & Reproductive Health Care. 2016; 21(4):265-268 and Rutgeerts, et al. “Effects of Bowel Mobility of Misoprostol Administered Before and After Meals.” Alimentary Pharmacology and Therapeutics 1991; 5(5): 533-542. Due to this claimed decrease in side effects, the approved product labelling for misoprostol oral tablets specifies that it should be taken with food to decrease the side effects.

The prior art also suggests that oral tablets have significant oral irritation when given buccally and sublingually. Schaff E A, DiCenzo R, Fielding S L. Comparison of Misoprostol Plasma Concentrations Following Buccal and Sublingual Administration. Contraception. 2005; 71(1):22-25.

Due to this claimed decrease in side effects, the approved product labelling for misoprostol oral tablets specifies that it should be taken with food to decrease the side effects. The prior art also suggests that oral tablets have significant oral irritation when given sublingually. Schaff E A, DiCenzo R, Fielding S L. Comparison of Misoprostol Plasma Concentrations Following Buccal and Sublingual Administration. Contraception. 2005; 71(1):22-25.

Variable disintegration rates may also be problematic particularly in non-oral administration. Patients may find it difficult to keep the tablet and its fragments in place until the dosage form is fully disintegrated which may adversely impact absorption. Moreover, with extended disintegration times, fragments may migrate, for example, from sublingual space and may even be swallowed. With vaginal or rectal administration fragments or even intact tablets may fall out, or migrate to different zones in the cavity with different absorption characteristics. The variability in absorption may be further magnified in vaginal and rectal delivery routes where variable or extended disintegration and dispersion may be more of a problem due to low mucosa moisture exposure as compared to sublingual moisture exposure in the mouth. Thus, a formulation having consistently rapid disintegration, for non-oral administration would be of clinical importance for patient care.

In addition, misoprostol is known to degrade rapidly when exposed to moisture. This can occur with relatively minor exposure such as following opening of large pharmaceutical bottles (typically containing 60-120 tablets) for dispensing smaller quantities to patients (often 1 to 12 tablets). The exposure over time with tablets supplied in large format bottles may result in variable potency. An alternative may be to provide misoprostol in a blister pack format where each tablet is individually packaged in material impermeable to air or moisture. The process of blistering individual tablets, however, may stress on tablets and increase breaking and chipping, which in turn introduces further variability to the dose actually administered. Increasing tablet hardness may result in tablets that are slower to disintegrate, dissolve and thus have lower and more variable absorption kinetics. Thus, it is clinically desirable to have a dosage form for non-oral route that has a higher level of hardness, sufficient to protect it during packaging and handling even in a blister pack, but that retains rapid consistent disintegration, dissolution and absorption.

### SUMMARY

There is a need for a dosage form of misoprostol that is an alternative to oral dosing and is suitable for non-oral dosing. The dosage form may desirably maintain a consistently rapid disintegration and dissolution time with a higher level of hardness to ensure the dosage form does not fragment and is able to better withstand handling and packaging operations, especially blister packaging. To this end, a dispersible solid pharmaceutical composition non-oral dosage form is provided that results in a better pharmacokinetic profile in conjunction with a reduced incidence of adverse side effects and may be administered under fasting conditions. The non-oral dosage form may also have reduced oral irritation as compared to oral forms. The non-oral dosage form may include misoprostol or a pharmaceutically acceptable salt thereof, one or more disintegrants, lubricants, diluents, and optionally one or more other excipients (including but not limited or antioxidant, color, or flavor). The non-oral dosage form may have a hardness of about 6 to about 10 kp, allows administration non-orally, namely sublingually, vaginally or rectally; disintegration or dispersion in less than about 60 seconds; and has a greater than about 80 percent dissolution at about 15 minutes. It may be in the form of a tablet or other solid dosage form as known to practitioners of the art.

Furthermore, counterintuitive to art recommended regiment of oral tablets being administered in a fed state, the dosage form of the invention administered by non-oral routes such as sublingual to a fasting subject surprisingly provides a superior pharmacokinetic and safety profile relative to the profile obtained when administered with food. This is in sharp contrast to what is taught or suggested in the prior art such as in Rutgeerts, et al. “Effects of Bowel Mobility of Misoprostol Administered Before and After Meals.” in which it is suggested that misoprostol be administered with food. The AUCs may be similar between fasting and fed, but the Cmax of in a fasting subject may be much higher, and the elimination phase may be much more rapid when administered fasting. The variability of pK parameters (% CV) may also be reduced. However, surprisingly, the incidence of adverse effects may be approximately half between fasting and fed with our invention. Thus, the onset of effects to a patient may be quicker, more complete, and may be cleared from the body much quicker without inducing elevated adverse events.

The dosage form of the invention may be administered either fed or fasting conditions, but preferably in a fasting form. Such administration may be in which the patient has not taken food other than clear liquids within less than about 2 hrs before administration, about 4 hrs before administration, about 6 hrs before administration, about 8 hrs before administration, or about 10 hrs before administration.

The dosage form may include misoprostol or a pharmaceutically acceptable salt thereof, a disintegrant, a lubricant, a diluent, and optionally one or more excipients. The formulation may be administered to the subject as a dosage form having a content of about 10 to about 1000 mcg misoprostol or equivalent amount of a pharmaceutically acceptable salt thereof, and allows disintegration or dispersion in less than about 60 seconds and has a greater than about 80 percent dissolution at 15 minutes.

### DESCRIPTION OF THE DRAWINGS

FIG. 1 is a main plasma concentration versus time curve for the formulation of Example 1.

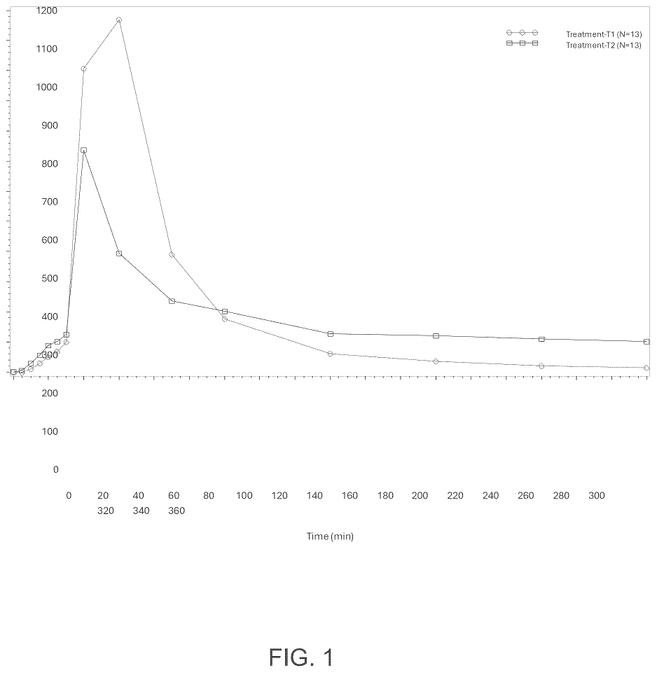

### DETAILED DESCRIPTION

The foregoing and other aspects of the present disclosure will now be described in more detail with respect to the description and methodologies provided herein. It should be appreciated that the disclosure can be embodied in different forms and should not be construed as limited to the embodiments set forth herein. Rather, these embodiments are provided so that this disclosure will be thorough and complete, and will fully convey the scope of the disclosure to those skilled in the art.

The terminology used in the description of the disclosure herein is for the purpose of describing particular embodiments only and is not intended to be limiting of the disclosure. As used in the description of the embodiments of the disclosure and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items.

The term “about,” as used herein when referring to a measurable value such as an amount of a compound, dose, time, temperature, and the like, is meant to encompass variations of 20 percent, 10 percent, 5 percent, 1 percent, 0.5 percent, or even 0.1 percent of the specified amount. Unless otherwise defined, all terms, including technical and scientific terms used in the description, have the same meaning as commonly understood by one of ordinary skill in the art to which this invention belongs.

As used herein, the terms “comprise,” “comprises,” “comprising,” “include,” “includes” and “including” specify the presence of stated features, integers, steps, operations, elements, and/or components, but do not preclude the presence or addition of one or more other features, integers, steps, operations, elements, components, and/or groups thereof.

All patents, patent applications and publications referred to herein are incorporated by reference in their entirety. In case of a conflict in terminology, the present specification is controlling.

It is noted that any one or more aspects or features described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

The present disclosure is related to a dispersible solid pharmaceutical composition dosage form of misoprostol. The dosage form may include misoprostol or a pharmaceutically acceptable salt thereof as a dispersion on an inert carrier, a disintegrant, a lubricant, a diluent, and optionally one or more excipients. The dosage form may have a hardness of about 6 to 10 kp and allows administration non-orally, namely sublingually, vaginally or rectally, to disintegrate or disperse in less than about 60 seconds and has a greater than about 80 percent dissolution at about 15 minutes. Rapid disintegration may result in less variability of absorption. Additionally, rapid disintegration may result in less fragmentation. Fragments may fall out or migrate depending on the method of non-orally administering. The dosage form may have minimum adverse events including minimum drug/food interaction.

The term “pharmaceutically acceptable” refers to compounds or compositions which are, within the scope of sound medical judgment, suitable for contact with the tissues of human beings and animals without excessive toxicity, irritation, allergic response, or other problem complications commensurate with a reasonable benefit/risk ratio.

The present disclosure provides a dispersible solid pharmaceutical composition that may be administered non-orally. For example, the dispersible solid pharmaceutical composition of the invention may be administered by sublingual diffusion through tissues in the gum and cheeks, by vaginal insertion, or by rectal insertion. This is in contrast to oral administration in which disintegration, dissolution, diffusion and absorption occurs in the stomach and small intestine, with the absorbed drug then traveling to the liver before entering circulation. The liver may extensively metabolize and alter the drug. This is known as “first-pass effect” of the drug metabolism.

The non-oral administration of misoprostol of the invention may provide a faster and more effective administration thereof leading to better efficiency and fewer adverse side events. This advantage may be further optimized by the invention by providing rapid disintegration and dispersion. There may be less adverse events by reducing exposure in the GI tract due to also avoiding GI first-pass effects. If administered under fasted conditions, Cmax may be elevated with non-significant changes to Tmax or AUC and there is also a lower variability to Cmax and Tmax in the fasted state which should provide a more consisted dose-response relationship in vivo leading to more consistent bioavailability and therapeutic effects while reducing the percentage of adverse events, particularly adverse food events. The sublingual dosage form may have reduced instance of oral irritation.

Misoprostol tablets may be prepared with an about 1 percent dispersion of misoprostol on an inert substrate such as hydroxypropylmethylcellulose. The dosage form may include about 0.5 to about 20 percent of the dispersion by weight containing misoprostol or pharmaceutically acceptable salt thereof, about 1 to about 10 percent by weight disintegrant, about 0.1 to about 2.0 percent by weight lubricant, about 20 to about 90 percent by weight diluent; and about 0 to about 30 percent one or more excipients. Alternatively, the dosage form may be prepared with other dispersions/preparations of misoprostol, including dispersions containing up to about 10 percent misoprostol. The dosage form in one embodiment may be in the form of a tablet, capsule and the like.

Suitable disintegrants may include, but are not limited to, alginic acid, alginates, β-cyclodextrin, sodium starch glycolate, corn starch and the like and blends and mixtures thereof. In one embodiment, the disintegrant may be present in an amount of about 2 to about 10 percent by weight. Disintegration may occur by rapid uptake of water followed by rapid swelling. In another embodiment, the disintegrant may be Glycols® Type-A sodium starch glycolate from Roquette.

Suitable lubricants may include, but are not limited to, hydrogenated castor oil, magnesium stearate, calcium stearate, sodium benzoate, talc, silica, and the like. In operation, the lubricant may be used as a coating or formed as a solid matrix. The lubricant may be present in an amount of about 0.1 to about 2.0 percent by weight. In one embodiment, hydrogenated castor oil, a hard wax with a high melting point available from BASF may be used.

Suitable diluents may include, but are not limited to, lactose, mannitol, xylitol, microcrystalline cellulose, sugar, dextrin, hydrophilic carbonate and the like, and blends and mixtures thereof. In one embodiment the diluent is microcrystalline cellulose. In one embodiment, the diluent may be present in an amount of about 20 to about 90 percent by weight. Often larger particle size diluents may be used to provide better flow and reduced moisture levels. The cellulose-based diluents may have a molecular weight grade of about 90,000 MN to about 1,500,000 MN. In another embodiment, microcrystalline cellulose such as Avicel PH 112 available from IFF may be used. Avicel PH 112 has a particle size of about 100 μm.

Exemplary excipients may include wetting agents (e.g., polysorbates, lauryl sulfate and lecithin), coloring agents, sweeteners, oils, preservatives, antioxidants, buffering agents and the like, known to those skilled in the art.

The pharmaceutical composition may include a coating. The coating may include but is not limited to, hydroxypropyl methylcellulose (HPMC, methocel), hydroxymethyl cellulose, polyvinylpyrrolidone (PVP), polyethylene glycol, dextrin maltodextrin, polyvinyl acetate (PVA) based compounds, hydroxypropyl cellulose (HPC), cellulose acetate, cellulose phthalate, derivatives thereof, or combinations thereof.

The pharmaceutical composition may be used in combination, concomitantly contemporaneously or in a sequential regimen, in the same formulation or in separately administered appropriate dosage form. Such other active ingredients include but are not limited to antiprogestins (including but not limited to mifepristone and ulipristal acetate), antimetabolite/antifolates (including but not limited to methotrexate), and aromatase inhibitors (including but not limited to letrozole). Mifepristone is 11 β-hydroxy-17-(1-proy-nl) estra-4, 9-diene-3-one. The combination approach with other active ingredients may include methods for separate, for simultaneous, or for sequential use in the treatment, or in the treatment of an obstetric or gynecological condition based on need and timing.

The pharmaceutical composition of the invention may be administered non-orally, namely sublingually, vaginally or rectally to treat a variety of obstetric and/or gynecological conditions including inducing cervical ripening, inducing uterine contractions, induction of pregnancy, resolution of miscarriage, and termination of pregnancy. The improvement in the administration of a dosage form of misoprostol may be using a dosage form having a hardness of about 6 kp to about 10 kp is that the dosage form may disintegrate or disperse in less than 60 seconds and has a greater than about 80 percent dissolution at 15 minutes. Moreover, such dosage form is preferably given in a fasting condition which is contrary to the recommendation to administer oral form in a fed condition. The pharmaceutical composition of the invention may reduce variability of absorption due to reduced disintegration time and the consistency of such absorption, particularly when taken under fasting conditions.

The following examples are merely illustrative of the invention and disclosure and are not limited thereto.

### Example 1

Tablets conforming to the above invention were prepared as follows:

| | | 200 mcg tablet |
|---|---|---|
| Ingredient | % W/W | (mg/tab) |
| Misoprostol (in a 1% HPMC dispersion) | 10.10 | 20.20 |
| Microcrystalline cellulose | 85.80 | 171.60 |
| Sodium starch glycolate | 3.60 | 7.20 |
| Hydrogenated Castor Oil | 0.50 | 1.000 |

Physical attributes such as weight variation, hardness and thickness of the finished product were well within the predefined specification.

### Examples 2 and 3

### Fasted and Fed

To test bioavailability of the above invented formulation and manufacture, subjects were tested by a single sublingual administration. Testing was performed in a two group sequential crossover design measuring blood levels of misoprostol acid.

| | | | |
|---|---|---|---|
| | Tmax | Cmax | AUC0-t |
| Measure | (min) | (pg/mL) | (pg · h/mL) |

Treatment Product-Fasted SL

| | | | |
|---|---|---|---|
| N | 14 | 14 | 14 |
| Mean | 39.8 | 2194 | 2235 |
| SD | 15.2 | 1044 | 712 |
| CV (%) | 38.1 | 47.6 | 31.9 |

Treatment Product-Fed SL

| | | | |
|---|---|---|---|
| N | 14.0 | 14 | 14 |
| Mean | 33.6 | 2072 | 2468 |
| SD | 9.1 | 979 | 916 |
| CV (%) | 17.0 | 47.3 | 37.1 |

We have discovered that our formulation, which has a shorter, consistent disintegration rate, with sublingual administration to fasted subjects provided a higher absorption with more consistency (Cmax, % CV) and more consistent Tmax with no adverse gastrointestinal events in the fasted or fed population and no oral irritation. Only one adverse event was seen in this study, which was pyrexia, likely due to the higher Cmax levels seen. This effect is particularly unexpected, given that our formulation compared to the prior art (Karim oral 200 mcg dose) has much higher Cmax (2194 pg/mL vs 811) and AUC (2234 pg·hr/mL (0-6 hr) vs 417 pg·hr/mL (0-4 hr)) but with a more controlled rise to Cmax (50 min vs 14 min). Our formulation compared to the prior art formulation (Schaff 800 mcg doses) also shows a higher Cmax (2194 vs 400 pg/mL) and AUC (2234 pg·hr/mL (0-6 hr) vs 380 pg·hr/mL (0-4 hr) more controlled rise Tmax (48 min vs 30 min). Our invention with its higher, but more controlled absorption, with lesser side effects, provides a more beneficial formulation for non-oral administration. The favorable effect of administering in a fasted condition is contrary to prior teaching. Such as for example, in Rutgeerts, et al. “Effects of Bowel Mobility of Misoprostol Administered Before and After Meals.” Alimentary Pharmacology and Therapeutics Rutgeerts, reported that receiving 400 or 800 mcg daily resulted in GI adverse events such as diarrhea and abdominal pain. Therein, a dose of 400 mcg was administered fasted and fed for twelve patients for five (5) days with the following results shown In Table 2:

Dose 400 mcg oral bid fasted and fed, administered for five days, 12 subjects. (Rutgeerts, et al)

TABLE 2

| | Fasted | Fed | Placebo |
|---|---|---|---|
| Flatulence | 6 | 5 | 1 |
| Ab Pain | 6 | 4 | 1 |
| Nausea | 4 | 1 | 1 |
| Diarrhoea | 2 | 1 | 0 |
| Bowel irregularity | 0 | 0 | 1 |
| Fever | 0 | 0 | 1 |
| Rhinitis | 1 | 0 | 0 |
| Vomiting | 1 | 0 | 0 |
| Total | 20 | 11 | 5 |

Most notably as in the above Table 2, with the prior art oral formulations, administration under fasting conditions resulted in more than twice the number of adverse events than was observed in Fed subjects (20 vs 11). By contrast, with twice the misoprostol dose (800 mcg) formulated with our invention, sublingual administration resulted in none of the adverse events observed in Rugeerts with 400 mcg (14 vs 20) as shown in Table 3:

TABLE 3

| | Fasted | Fed |
|---|---|---|
| Flatulence | 0 | 2 |
| Ab Pain | 0 | 0 |
| Nausea | 0 | 0 |
| Diarrhea | 0 | 0 |
| Bowel irregularity | 0 | 0 |
| Fever | 0 | 0 |
| Nasopharyngitis | 0 | 0 |
| Vomiting | 0 | 0 |
| Back Pain | 0 | 0 |
| Dizziness | 0 | 0 |
| Pyrexia | 1 | 0 |
| Menstrual | 0 | 0 |
| Irregularity | | |
| Total | 1 | 0 |

The above fed/fasting study on the formulation of the invention demonstrate the unexpected result of the present formulation being able to be taken under fasting or fed conditions with no adverse gastrointestinal events which is directly contrary to oral administration and the need to administer in a fed condition. Furthermore, no oral irritation was observed in any patient under fed or fasted conditions in contrast to prior art formulations.

Although the present approach has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present approach.

That which is claim is:

1. A dispersible solid pharmaceutical composition non-oral dosage form comprising:
(a) misoprostol or a pharmaceutically acceptable salt thereof;
(b) a disintegrant;
(c) a lubricant;
(d) a diluent; and,
(e) optionally one or more excipients, wherein the dosage form has a hardness from about 6 to about 10 kp and allows non-oral administration and the dosage form to disintegrate or disperse in less than about 60 seconds and has a greater than about 80 percent dissolution at 15 minutes.

2. The dispersible solid pharmaceutical dosage form of claim 1, wherein the disintegrant is selected from the group consisting of sodium starch glycolate, crospovidone, croscarmellose sodium, and B-cyclodextrin.

3. The dispersible solid pharmaceutical dosage form of claim 1, wherein the lubricant is selected from the group consisting of hydrogenated castor oil, magnesium stearate, calcium stearate, sodium benzoate talc, and silica.

4. The dispersible solid pharmaceutical dosage form of claim 1, wherein the diluent is microcrystalline cellulose.

5. The dispersible solid pharmaceutical dosage form of claim 1, wherein the dosage form comprises about 0.5 percent to about 20 percent by weight dispersion of misoprostol.

6. The dispersible solid pharmaceutical non-oral dosage form of claim 1, wherein the non-oral dosage when administered under fasting conditions demonstrates minimal oral irritation and fewer adverse events than under fed conditions.

7. The dispersible solid pharmaceutical non-oral dosage form of claim 1, wherein the non-oral dosage when administered at a dose of 800 mcg under fasting conditions demonstrates substantially elevated Cmax of at least about 100 μg/ml of blood levels of misoprostol acid than when administered under fed conditions.

8. A method of administering a non-oral dosage form for treatment of a condition wherein misoprostol is indicated, the non-oral dosage form comprising solid pharmaceutical composition non-oral dosage form comprising misoprostol or a pharmaceutically acceptable salt thereof; a disintegrant; a lubricant; a diluent; and, optionally one or more excipients, wherein the non-oral dosage form has a hardness from about 6 to about 10 kp and allows non-oral administration and the non-oral dosage form to disintegrate or disperse in less than about 60 seconds and has a greater than about 80 percent dissolution at 15 minutes.

9. The dispersible solid pharmaceutical non-oral dosage form of claim 8, wherein the disintegrant is selected from the group consisting of crospovidone, alginic acid, alginates, β-cyclodextrin, sodium starch glycolate, corn starch and blends and mixtures thereof.

10. The method of claim 8, wherein the lubricant is selected from the group consisting of hydrogenated castor oil, magnesium stearate, calcium stearate, sodium benzoate talc and silica.

11. The method of claim 8, wherein the diluent is microcrystalline cellulose.

12. The method of claim 8, wherein the non-oral dosage form comprises about 0.5 to about 20 percent by weight dispersion of misoprostol.

13. The method of claim 8, wherein Cmax is elevated and Tmax and AUC remain substantially the same.

14. The method of claim 8, wherein administering the non-oral dosage form comprises administering about 10 to about 1000 mcg of the non-oral dosage form.

15. The method of claim 8, wherein the non-oral dosage when administered under fasting conditions demonstrates minimal oral irritation and fewer adverse events than under fed conditions.

16. The method of claim 8, wherein the non-oral dosage when administered at a dose of 800 mcg under fasting conditions demonstrates substantially elevated Cmax of at least about 100 μg/ml of blood levels of misoprostol acid than when administered under fed conditions.

17. In a method of treating various obstetric or gynecological conditions of a patient in need thereof with a pharmaceutically acceptable solid misoprostol composition or pharmaceutically acceptable salt thereof in non-oral dosage form, the non-oral dosage form having a hardness from about 6 to about 10 kp and when administered non-orally disintegrates or disperses in less than about 60 second and has a greater than about 80 percent dissolution at 15 minutes, the improvement comprising the elevation of Cmax, Tmax and AUC remaining the same and less adverse events as compared to misoprostol or salts thereof given non-orally under fed conditions.

18. The method of claim 17, wherein there are reduced oral irritations.

19. The method of claim 17 wherein the misoprostol non-oral dosage form further includes a disintegrant, a lubricant, a diluent and one or more excipients.

20. The method of claim 19, wherein the disintegrant is selected from the group consisting of crospovidone, alginic acid, alginates, β-cyclodextrin, sodium starch glycolate, corn starch and blends and mixtures thereof.

21. The method of claim 19, wherein the lubricant is selected from the group consisting of hydrogenated castor oil, magnesium stearate, calcium stearate, sodium benzoate talc, and silica.

22. The method of claim 19, wherein the non-oral dosage form comprises about 0.5 to about 20 percent by weight dispersion of misoprostol.

23. The method of claim 17, wherein administering the non-oral dosage form comprises administering about 10 to about 1000 mcg of the non-oral dosage form.

24. The method of claim 17 in a regimen wherein said misoprostol non-oral dosage form is used in a regimen with one of the following agents, wherein said misoprostol and other agents are in the same or a separate non-oral dosage form and the agent is selected from the group consisting of antiprogestins, antimetabolites, antifolates and aromatase inhibitors.

25. A method of claim 24 wherein said other agent is mifepristone or ulipristal acectate.

26. A method of claim 24 wherein said other agent is methotrexate.

27. A method of claim 24 wherein said other agent is letrozole.
